(19) United States
(12) Patent Application Publication
(10) Pub. No.: US 2001/0007043 A1
(43) Pub. Date: Jul. 5, 2001

(54) CONTINUOUS RECOVERY OF (METH)ACRYLIC ACID

(76) Inventors: OTTO MACHHAMMER, MANNHEIM (DE); SUSANNE HAUPT, OFFENBACH (DE); VOLKER SCHLIEPHAKE, SCHIFFERSTADT (DE); JURGEN SCHRODER, LUDWIGSHAFEN (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 09/362,913

(22) Filed: Jul. 29, 1999

(30) Foreign Application Priority Data
Aug. 26, 1998 (DE) 19838817.9

(51) Int. Cl. C07C 69/74; C07C 51/42

(52) U.S. Cl. 562/600

(57) ABSTRACT

(Meth)acrylic acid is recovered continuously from the reaction gas containing (meth)acrylic acid, low boilers, medium boilers and high boilers and originating from a catalytic gas-phase oxidation by

(I) quenching the reaction gas by evaporative cooling with a high-boiling organic solvent,

(II) isolating the (meth)acrylic acid from the quenched reaction gas by absorption into the high-boiling organic solvent and

(III) separating the organic solvent laden with (meth)acrylic acid into a first part-stream (IIIA), which contains predominantly (meth)acrylic acid, and into a second part-stream (IIIB), which contains predominantly the solvent, by a process in which

(IV) part-stream IIIB is stripped free of (meth)acrylic acid with inert gas,

(V) the purified solvent from part-stream IIIB is recycled to the absorption stage II and

(VI) (meth)acrylic acid is obtained from part-stream IIIA by distillation, all liquid residual streams obtained in stage VI being recycled to the quench stage I.

The present invention relates to a process for the continuous recovery of (meth)acrylic acid by absorption of (meth)acrylic acid from the reaction gases of a catalytic gas-phase oxidation. Below, the term (meth)acrylic acid represents substances acrylic acid and/or methacrylic acid.

(Meth)acrylic acid is prepared predominantly by catalytic gas-phase oxidation of suitable starting materials, in particular of propene and/or acrolein in the case of acrylic acid and of isobutene and/or methacrolein in the case of methacrylic acid.

A number of possibilities are known for isolating the (meth)acrylic acid from the reaction gases of the catalytic gas-phase oxidation, including isolation by absorption in a solvent.

DE-B 21 36 396 discloses the isolation of the acrylic acid from the reaction gases obtained in the catalytic oxidation of propene or acrolein by countercurrent absorption with a mixture of 75% by weight of diphenyl ether and 25% by weight of biphenyl. Furthermore, DE-A 24 49 780 discloses the cooling of the hot reaction gas by partial evaporation of the solvent in a direct condenser (quench apparatus) before the countercurrent absorption. The problem here and in further process steps, in particular in the purification of the (meth)acrylic acid by distillation, is the production of solids in the apparatuses, which reduces the availability of the plant. According to DE-A 43 08 087, this solid fraction can be reduced in the case of acrylic acid by adding a polar solvent, such as dimethyl phthalate, in an amount of from 0.1 to 25% by weight, to the relatively nonpolar solvent mixture comprising diphenyl ether and biphenyl; this increases the absorptivity of the solvent mixture for the contaminants. However, with increasing polarity, the solvent absorbs increasing amounts of water; moreover, this leads to higher solvent losses via the dilute acid solution.

In the presence of solvents, at relatively high temperatures as occur in the recovery of (meth)acrylic acid by the process of the generic type, in particular on the bottom collecting tray of the absorption column, in the stripping section and in the bottom section of the distillation column and in the heat exchangers, the polyacrylic acid forms contamination which adheres firmly to the surface of the apparatuses and can be detached only with alkalis. Analyses have shown that the contamination comprises a mixture of from about 10 to 50% by weight of poly(meth)acrylic acid, the remainder being solvent.

It is an object of the present invention substantially to avoid the susceptibility to soiling in all apparatuses, in particular the production of contamination only soluble in alkali, and hence to improve the availability of the plant and the cost-efficiency of the process for the recovery of (meth)acrylic acid

We have found that this object is achieved by a process for the continuous recovery of (meth)acrylic acid from the reaction gas containing (meth)acrylic acid, low boilers, medium boilers and high boilers and originating from a catalytic gas-phase oxidation by

(I) quenching the reaction gas by evaporative cooling with a high-boiling organic solvent,

(II) isolating the (meth)acrylic acid from the quenched reaction gas by absorption into the high-boiling organic solvent and

(III) separating the organic solvent laden with (meth)acrylic acid into a first part-stream (IIIA), which contains predominantly (meth)acrylic acid, and into a second part-stream (IIIB) which contains predominantly the solvent.

In the process,

(IV)part-stream IIIB is stripped free of (meth)acrylic acid with an inert gas,

(V) the purified solvent from part-stream IIIB is recycled to the absorption stage II and

(VI)(meth)acrylic acid is recovered from part-stream IIIA by distillation, all liquid residual streams obtained in stage VI being recycled to the quench stage I.

Preferably, the temperature in each process stage does not exceed 155°C., in particular does not exceed 140°C., particularly preferably does not exceed 120°C.

Here, the term liquid residual streams denotes all liquid streams obtained in the process, except for the main product stream, for example the part-streams (a) and (c) described in more detail below.

It was found that oligomers are formed in the recovery of (meth)acrylic acid by distillation (stage VI) and were to date entrained via the liquid residual streams into upstream apparatuses. The invention avoids recycling the liquid residual streams from the distillative recovery to the liquid recycled solvent, and can therefore substantially inhibit soiling of the upstream apparatuses. The production of oligomers and hence the production of solid in the apparatuses upstream of process stage VI are lower in the novel process; the dual-flow or valve trays necessary to date in process stages II and IV according to the conventional processes can therefore be replaced by more highly hydrodynamically loadable baffles, for example dumped or arrangement packings. The substantial avoidance of soiling by the novel flow arrangement has far-ranging economic consequences: in particular, it is possible to use in the apparatuses elements such as dumped packings or stacked packings which have a greater hydrodynamic load capacity but also higher susceptibility to soiling compared with, for example, dual-flow or valve trays, which are used in the known process for isolating (meth)acrylic acid from mixtures in a high-boiling solvent, owing to their lower susceptibility to soiling. New plants can thus be dimensioned with smaller separation apparatuses, in particular columns, for the same level of production, or the level of production is increased by the novel process in existing plants.

Here, solvents whose boiling point is higher than the boiling point of the respective desired main product (about 141°C. for acrylic acid or about 161°C. for methacrylic acid, in each case at atmospheric pressure) are defined as high-boiling.

Starting mixtures for the present process are the reaction gases from the catalytic gas-phase oxidation of C₃-alkanes, C₃-alkenes, C₃-alkanols and/or C₃-alkanes, C₃-alkenes, C₃-alkanols and/or C₃-alkanals or intermediates for these, to give methacrylic acid. The process is described below for acrylic acid but is it also applicable in an analogous manner for methacrylic acid.

The catalytic gas-phase oxidation of propene and/or acrolein to acrylic acid in air or molecular oxygen by known processes, in particular as described in the abovementioned publications, is particularly advantageous. Temperatures of 200 to 450°C. and, if required, superatmospheric pressure are preferably employed here. The heterogeneous catalysts used are preferably oxidic multicomponent catalysts based on the oxides of molybdenum, bismuth and iron in the 1st stage (oxidation of propene to acrolein) and of the oxides of molybdenum and vanadium in the 2nd stage (oxidation of acrolein to acrylic acid). If propane is used as starting material, it can be converted into a propene/propane mixture by catalytic oxydehydrogenation as described in U.S. Pat. No. 5,510,558 or by homogeneous oxydehydrogenation, corresponding to the example in EP-A-0 253 409. When a propene/propane mixture is used, propane acts as a diluent gas. Suitable propene/propane mixtures are also refinery propene (70% of propene and 30% of propane) or cracker propene (95% of propene and 5% of propane). In principle, propene/propane mixtures containing oxygen and nitrogen in any composition can be oxidized to acrolein and acrylic acid.

The conversion of propene to acrylic acid is highly exothermic. The reaction gas which, in addition to the starting materials and products, advantageously contains an inert diluent gas, for example recycled gas (see below), atmospheric nitrogen, one or more saturated C₁- to C₆-hydrocarbons, in particular methane and/or propane, and/or steam can therefore absorb only a small part of the heat of reaction. Although the type of reactor used is not subject to any restriction per se, tube-bundle heat exchangers which are cooled by means of a salt bath and are filled with the oxidation catalyst are generally used since the heat evolved in the reaction can be very readily dissipated therein by convection and radiation to the cooled tube walls.

In the case of the catalytic gas-phase oxidation, it is not pure acrylic acid which is obtained but a gaseous mixture which, in addition to the acrylic acid, may contain essentially unconverted acrolein and/or propene, steam, carbon monoxide, carbon dioxide, nitrogen, propane, oxygen, acetic acid, propionic acid, formaldehyde, further acids and aldehydes, maleic acid and maleic anhydride as secondary components. Usually, the reaction product mixture contains from 1 to 30% by weight of acrylic acid, from 0.05 to 1% by weight of propene and from 0.05 to 1% by weight of acrolein, from 0.05 to 10% by weight of oxygen, from 0.05 to 2% by weight of acetic acid, from 0.01 to 2% by weight of propionic acid, from 0.05 to 1% by weight of formaldehyde, from 0.05 to 2% by weight of aldehydes, from 0.01 to 0.5% by weight of the sum of maleic acid and maleic anhydride and from 20 to 90, preferably from 50 to 98, % by weight of inert diluent gases, based in each case on the total reaction mixture. In particular, saturated C₁-C₆-hydrocarbons, such as from 0 to 95% by weight of methane and/or propane, as well as from 1 to 30% by weight of steam, from 0.05 to 15% by weight of carbon oxides and from 0 to 95% by weight of nitrogen, based in each case on 100% by weight of reaction gas, are present as inert diluent gases.

The process stages for isolating the acrylic acid from the reaction mixture are described below:

Stage I

The hot reaction gas is cooled by partial evaporation of the solvent in a direct condenser or quench apparatus prior to the absorption. Venturi scrubbers, bubble columns or spray condensers are particularly suitable for this purpose. The high-boiling secondary components of the reaction gas condense into the unevaporated solvent. In addition, the partial evaporation of the solvent is a purification step of the solvent. In a preferred embodiment of the invention, a part-stream of the unevaporated solvent, preferably from 1 to 10% of the mass flow fed to the absorption column, is removed and is subjected to a solvent purification. Here, the solvent is distilled over and the high-boiling secondary components remain behind and can be disposed of, e.g. incinerated, if required after further thickening. This solvent distillation serves for avoiding an excessively high concentration of high boilers in the solvent stream. The solvent distilled over is preferably fed to the laden solvent stream from the absorption column.

Stage II

In stage II, the acrylic acid and a part of the secondary components are separated from the reaction gas by absorption in a high-boiling solvent. Preferably, the boiling point of the high-boiling solvent is at least 20°C., in particular 50°C., more preferably 70°C., above the boiling point of the acrylic acid or methacrylic acid. Preferred solvents have boiling points (atmospheric pressure) of from 180 to 400°C., in particular from 220 to 360°C., in the present application the term solvent also including solvent mixtures. Suitable solvents are high-boiling, extremely hydrophobic solvents which contain no externally active polar groups, such as aliphatic or aromatic hydrocarbons, for example middle oil fractions from paraffin distillation, or ethers having bulky groups on the O atom, or mixtures thereof, a polar solvent, such as the 1,2-dimethyl phthalate disclosed in DE-A-43 08 087, advantageously being added thereto. Esters of benzoic acid and phthalic acid with straight-chain alkanols of 1 to 8 carbon atoms, such as n-butyl benzoate, methyl benzoate, ethyl benzoate, dimethyl phthalate and diethyl phthalate, and thermal oils, such as biphenyl, diphenyl ether and mixtures of biphenyl and diphenyl ether or their chlorine derivatives and triarylalkanes, e.g. 4-methyl-4′-benzyldiphenylmethane and its isomers 2-methyl-2′-benzyldiphenylmethane, 2-methyl-4′-benzyldiphenylmethane and 4-methyl-2′-benzyldiphenylmethane, and mixtures of such isomers are furthermore suitable.

A particularly perferred solvent is a solvent mixture comprising biphenyl and diphenyl ether, preferably in the azeotropic composition, in particular comprising about 25% by weight of biphenyl and about 75% by weight of diphenyl ether, based on 100% by weight of biphenyl and diphenyl ether, for example the commercially available Diphyl®. This solvent mixture preferably furthermore contains a polar solvent, such as dimethyl phthalate, in an amount of from 0.1 to 25% by weight, based on the total solvent mixture. This reduces the susceptibility of the plants to soiling.

Here, the terms high boilers, medium boilers and low boilers and corresponding adjectives denote, respectively, compounds which have a higher boiling point than acrylic acid (high boilers), those which have about the same boiling point as acrylic acid (medium boilers) and those which have a lower boiling point than acrylic acid (low boilers).

The absorption takes place in a countercurrent absorption column which is preferably equipped with dumped or stacked packings and into which solvent flows from above. The gaseous reaction product and any evaporated solvent from the quench apparatus are passed from below into the column and then cooled to absorption temperatures. The cooling is advantageously effected by cooling loops, i.e heated solvent is removed from the column, cooled in heat exchangers and fed again to a point above the take-off point of the column. After the absorption, all high boilers, the major part of the acrylic acid and a part of the low boilers are present in the solvent.

The remaining, unabsorbed reaction gas is further cooled in order to separate therefrom the condensable part of the low-boiling secondary components, in particular water, formaldehyde and acetic acid, by condensation. This condensate is referred to below as dilute acid solution. The remaining gas stream predominantly comprises nitrogen, carbon oxides and unconverted starting materials. Some of these are preferably fed back to the reaction stages as diluent gas, referred to below as recycled gas. The atmospheric nitrogen and part of the uncondensed secondary components are removed as waste gas and preferably incinerated.

Stages III and VI

In stage III, the acrylic acid together with the medium boilers and the last residue of low boilers is separated from the solvent, resulting in a first part-stream III A, which predominantly contains acrylic acid, and a second part-stream III B, which predominantly contains the solvent.

The isolation of the acrylic acid from the mixture with the solvent is preferably effected by partial evaporation. The partial evaporation is carried out at from 10 to 200 mbar and corresponding evaporation temperatures from 60 to 130°C., in particular at from 60 to 100 mbar and from 90 to 110°C. The energy for evaporating the part-stream III A is obtained as far as possible by cooling the reaction gas. The lacking residual energy is covered by vapor condensation. The evaporation gives rise to a vapor stream and a liquid stream. The vapor stream III A contains the major part of the acrylic acid, i.e. has acrylic acid concentrations of from about 70 to 95%, preferably from about 80 to 90%. The acrylic acid is purified therefrom in further process steps by distillation. The liquid stream III B from the evaporator contains predominantly the solvent and acrylic acid in a concentration of from about 5 to 15% by weight.

This liquid stream III B is then purified by stripping. The purification of the solvent is described below as process stage IV.

In a preferred embodiment, the recovery of the (meth)acrylic acid from part-stream III A by distillation is effected in the following process steps:

VI-I Separation of a residual stream (a), which, in addition to (meth)acrylic acid, contains the low boilers and a part of the medium boilers and a part of the high boilers, and of a part-stream (b), which is completely or virtually completely free of low boilers, and

VI-II recovery of the (meth)acrylic acid from the part-stream (b).

The isolation of the acrylic acid from stream III A (stage VI) is effected by distillation, it being possible in principle to use any distillation column. Advantageously, a column having dual-flow trays is used for this purpose.

The part-stream III A is condensed and runs downward through the column VI-I. Vapor, predominantly acrylic acid vapor, rises countercurrent from the bottom upward and thus strips the low boilers of the liquid so that the liquid stream (b) arriving at the bottom is virtually free of low boilers. On the other hand, the medium boilers and high boilers predominantly remain in the liquid during the stripping and reduce the tendency of the acrylic acid to polymerize during the stripping process.

A stream (a) rich in low boilers is then taken off at the top of the column after a partial condensation. However, since the stream still contains acrylic acid, the vapor stream from distillation stage VI is advantageously not discarded but condensed and cooled, after which the cold condensate is recycled to quench stage I or absorption stage II.

The preferred operating parameters in the descending stripping column are: Top pressure <200, in particular <100, particularly preferably <50, mbar, bottom temperature <140°C., in particular <120°C., particularly preferably <100°C., and

acrylic acid concentration in the bottom product from 5 to 15, particularly preferably from 8 to 12, % by weight.

The recovery of the acrylic acid from the part-stream (b) is preferably effected by separating the part-stream (b) into a first part-stream which contains crude acrylic acid and may, if required, be further purified and a part-stream (c). Process stage VI-II is preferably effected by distillation in an ascending stripping column. Preferably, the descending stripping column for process stage VI-I and the ascending stripping column for process stage VI-II have a common bottom. The part-stream (b) obtained as a result of process stage VI-I in the common bottom of descending and ascending stripping columns is separated in the ascending stripping column in process stage VI-II. A part-stream (c) which predominantly contains the solvent and which, if necessary after a purification, in particular by evaporation in a quench apparatus, is recirculated to the absorption stage is obtained in the bottom of the column. In the ascending stripping column, the vapor completely or virtually completely free of low boilers ascends, the medium boilers and high boilers being washed out of the vapor by the liquid reflux. The vapor is condensed at the top of the column, a part is taken off at the top as a product and the remainder is liquid reflux. The product is acrylic acid which is substantially free of low boilers, medium boilers and high boilers. This acrylic acid is referred to as crude acrylic acid.

The crude acrylic acid obtained in stage VI contains preferably from 98 to 99.8, in particular from 98.5 to 99.5, % by weight of acrylic acid and from 0.2 to 2, in particular from 0.5 to 1.5, % by weight of impurities, for example acetic acid, aldehydes and maleic anhydride, based in each case on the crude acrylic acid. If its purity requirements are not very high, this acrylic acid may be used as such for esterification.

In a preferred embodiment of the invention, the dilute acid solution, which may still contain dissolved acrylic acid, is treated by extraction with a small part-stream of the virtually acrylic acid-free solvent (from stage IV). The aqueous stream from the extraction with dilute acid solution can be concentrated, which may be necessary in particular if there are environmental requirements.

If an acrylic acid concentration of from 5 to 15, in particular from 8 to 12, % by weight in the bottom product is permitted, it would be possible to maintain a bottom temperature of <155°C. at a bottom pressure of about 250 mbar and thus dramatically to reduce the degree of soiling (degree of fouling).

Stages IV and V

Before the recycling to absorption stage I, the solvent stream must be purified to remove acrylic acid substantially, in order to be able to absorb acrylic acid again from the reaction gas of the gas-phase oxidation; the acrylic acid concentration in the solvent stream should not exceed 1, preferably 0.5, % by weight.

The solvent stream III B obtained in stage III may still contain relatively large amounts of acrylic acid, up to about 15% by weight. The reduction in the acrylic acid content of the solvent is effected by stripping with an inert gas or with an inert gas mixture, preferably with a part-stream of the recycled gas or only propane, if propane is used as a diluent gas. The stripping gas is particularly preferably a part-stream of the recycled gas. The stripping is effected at from about 1.1 to 2.0, preferably from 1.3 to 1.6, bar and at from about 80 to 120°C., preferably from 110 to 120°C. During the stripping, the solvent stream to be purified is fed in at the top of the column; it flows over the baffles toward the bottom. The stripping gas is passed into the bottom of the stripping column and flows countercurrent. While the stripping gas flows toward the top of the column, it absorbs acrylic acid from the liquid solvent stream so that a purified solvent stream which has an acrylic acid concentration of not more than 1, preferably not more than 0.5, % by weight can be taken off from the bottom of the stripping column. This substantially acrylic acid-free solvent can then be recirculated to the absorption stage (I).

The stripping recycled gas laden with acrylic acid is expediently recirculated to the stage in which the partial evaporation of the solvent is effected, or to the absorption column.

Preferably, the stripping recycled gas is recycled from process stage IV to the reaction gas before process stage II.

The invention is illustrated in more detail below with reference to a drawing and with reference to embodiments.

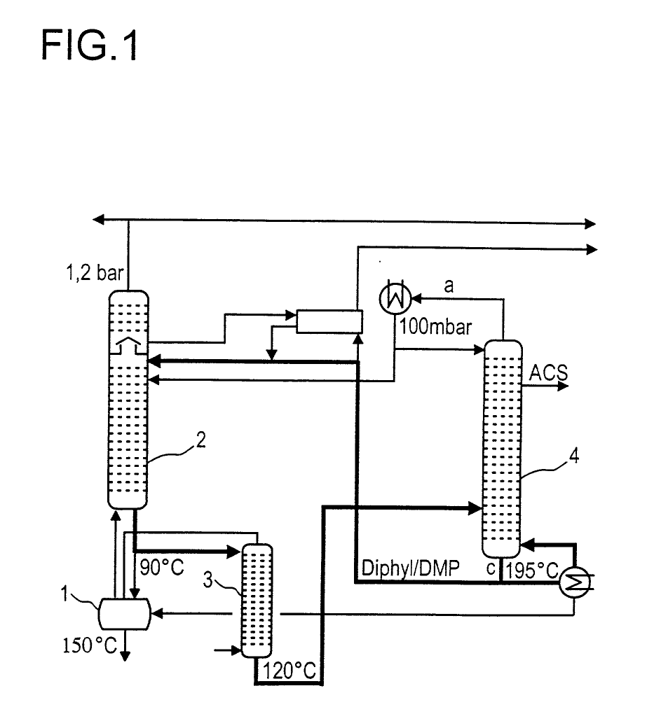

FIG. 1 shows the schematic representation of a plant according to the prior art and

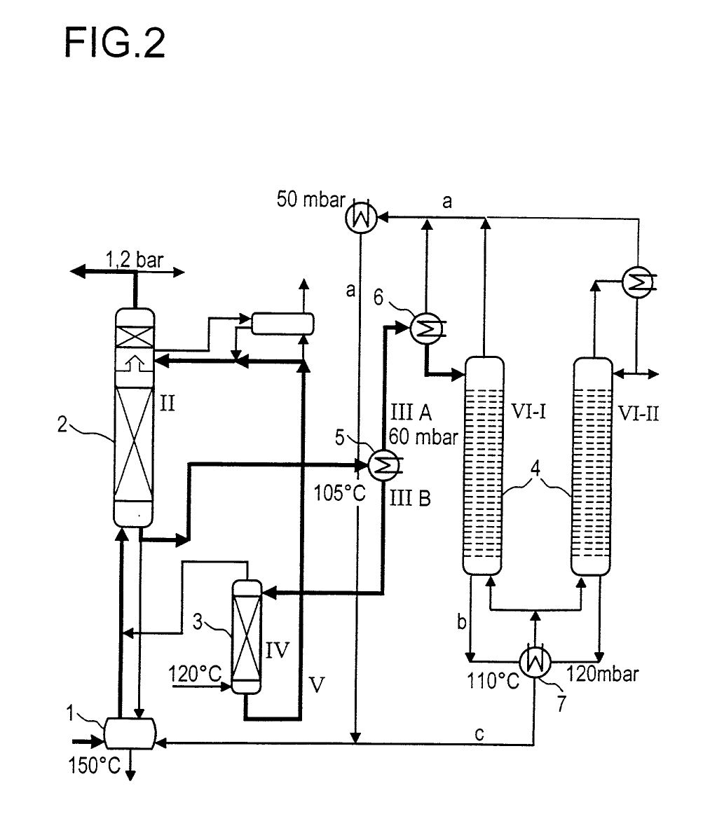

FIG. 2 shows the schematic representation of a plant according to the invention.

First, as a comparative example, the recovery of the acrylic acid by the conventional process in a plant corresponding to FIG. 1 will be described below:

2900 1(S.T.P.)/h of a gas stream from the gas-phase oxidation to acrylic acid, having a temperature of 270°C. and a pressure of 1.6 bar and comprising the main components (in each case in % by weight)

nitrogen (85),

acrylic acid (10) and

water (4)

were cooled to 150°C. in a Venturi quench apparatus 1 by direct contact with quench liquid (140-150°C.) which was sprayed in through slots provided in the region of the narrowest cross-section of the Venturi tube and which comprises 57.4% by weight of diphenyl ether, 20.7% by weight of biphenyl, 20% by weight of o-dimethyl phthalate, the remainder consisting of other components. That fraction of the quench liquid which had remained in the form of liquid drops was then separated from the gas phase consisting of reaction gas and vaporized quench liquid in a downstream mist separator (receiver having a gas tube leading upward) and recycled to the Venturi scrubber. A part-stream of the recycled quench liquid was subjected to a solvent distillation, the quench liquid being distilled over and high-boiling secondary components remaining behind and being incinerated.

The gas phase at about 150°C. was fed to the lower part of a packed absorption column 2 (3 m high; double jacket of glass; internal diameter of 50 mm; three packing zones 90 cm, 90 cm and 50 cm long (from bottom to top); the packing zones were thermostated as follows from bottom to top: 90°C., 60°C., 20°C.; the penultimate and the final packing zones were separated by a chimney tray; the packings were stainless steel coils having a coil diameter of 5 mm and a pitch of 5 mm; the absorbent was fed in directly above the middle packing zone and exposed to the countercurrent of 2900 g/h of the absorbent added at a temperature of 50°C. and likewise composed of 57.4% by weight of diphenyl ether, 20.7% by weight of biphenyl and 20% by weight of o-dimethyl phthalate, the remainder consisting of other components). The discharge of the absorption columns which, in addition to acrylic acid, also contained low-boiling byproducts, e.g. acrolein and acetic acid, in absorbed form, was indirectly heated to 100°C. in a heat exchanger and fed to the top of a desorption column 3 which was likewise designed as a packed column having a length of 2 m (double jacket of glass; 50 mm internal diameter; packing: stainless steel coils having a coil diameter of 5 mm and a pitch of 5 mm; one packing zone 1 m long; thermostated at 120°C.). In the desorption column 3, the components having a lower boiling point than acrylic acid, such as acrolein and acetic acid, were substantially removed from the acrylic acid/absorbent mixture by stripping with 600 1(S.T.P.)/h of air (countercurrent; feed temperature 120°C.). The stripping gas leaving the desorption column 3 was recirculated and was combined with hot reaction gas of the acrolein oxidation stage before its entry into the Venturi quench (stage I).

The unabsorbed gas mixture leaving the second packing zone in an upward direction in the absorption column 2 was further cooled in the third packing zone in order to separate off the condensable part of the secondary components contained therein, e.g. water and acetic acid, by condensation. This condensate is referred to as dilute acid solution. To increase the separation efficiency, a part of the dilute acid solution was recycled to the absorption column 2, above the third packing zone of the absorption column 2, at a temperature of 20°C. The dilute acid solution was taken off below the uppermost packing zone, from the chimney tray mounted there. The ratio of recycled to taken-off dilute acid solution was 200 g/g. The dilute acid solution removed also contained 0.8% by weight of acrylic acid in addition to 97.5% by weight of water. Said acrylic acid can, if required, be recovered as described in DE-A 196 00 955. 1600 1(S.T.P.)/h of the gas stream finally leaving the absorption column 2 were recycled to the propene oxidation. The remainder was incinerated.

The bottom liquid of the desorption column 3 was fed to the 8th tray from the bottom of a tray column 4 containing 57 dual-flow trays (internal diameter: 50 mm; length 3.8 m; top pressure: 100 mbar; bottom pressure: 280 mbar; bottom temperature: 195°C.; a pressure-drop resistance was provided at the 9th tray) and rectified in the tray column 4. At the 48th tray from the bottom, the crude acrylic acid was removed via a side take-off. The purity of the crude acrylic acid removed was ≧98% by weight. At the top of the rectification column 4, an acrylic acid-containing gas stream enriched with low boilers was taken off after a partial condensation (reflux ratio 8.7) and was recycled to the absorption column 2, above the lowermost packing zone. The absorbent free of low boilers and virtually free of acrylic acid was taken off from the bottom of the rectification column 4 and recycled to the absorption column 2, above the second packing zone (viewed from the bottom). A part-stream of 50 g/h was fed to the Venturi quench 1 for removing the high-boiling secondary components. Phenothiazine, as a polymerization inhibitor, was added to the reflux at the top of the rectification column 4, in amounts such that the side take-off contained 300 ppm of phenothiazine (a schematic diagram of the working-up process for the reaction gas of the acrolein oxidation stage is shown in DE-A 196 00 955; in addition, the working-up method is also described in DE-A 43 08 087).

An embodiment of the novel process is described below in conjunction with FIG. 2.

The procedure in the Venturi quench 1 and in the absorption column 2 corresponds to the prior art process described above in conjunction with FIG. 1. In contrast, the discharge of the absorption column 2 was fed not to the top of a desorption column 3 but to a forced-circulation flash evaporator 5 which was operated at 60 mbar and 105°C. An acrylic acid-laden solvent stream of 5230 g/h (main components, in each case in % by weight: solvent 61, acrylic acid 30, acetic aid 8118 ppm, maleic anhydride 2000 ppm) was separated into a first part-stream IIIA of 2160 g/h, which predominantly contained acrylic acid (main components, in each case in % by weight: solvent 20, acrylic acid 77 and acetic acid 0.22) and a second part-stream IIIB of 3070 g/h, which predominantly contained the solvent (main components, in each case in % by weight: solvent 83, acrylic acid 5 and acetic acid 636 ppm).

The part-stream IIIB was fed to the top of the stripping column 3. This was the same stripping column 3 as in the prior art process (FIG. 1) and the same stripping gas, i.e. an air stream of 600 1(S.T.P.)/h. However, the function of the stripping column 3 differed in the two processes, the prior art process on the one hand and the novel process on the other hand: in the prior art process (FIG. 1), the discharge from the absorption column 2 was fed to the top of the stripping column 3, and the stripping column 3 functioned as the desorption column. On the other hand, in the process according to the invention (FIG. 2), the part-stream IIIB from the evaporator was fed to the top of the stripping column 3; the stripping column 3 served here for removing acrylic acid from the solvent (process stage IV). The purified solvent was taken off from the bottom of the stripping column 3 and recirculated to the top of the absorption column 2.

The part-stream IIIA obtained in the evaporator 5 was condensed in a heat exchanger 6 at 100 mbar and the condensate was fed to the rectification column 4, now, in contrast to the prior art, divided into two at the 28th tray, specifically to the descending stripping section thereof In the descending stripping section of the rectification column 4, process stage VI-I took place, i.e. the low boilers were stripped from the part-stream IIIA by the countercurrent method with acrylic acid vapor, whereas the medium boilers and high boilers predominantly remained in the liquid. A stream b virtually free of low boilers (main components in % by weight: solvent 28, acrylic acid 71, acetic acid 721 ppm, maleic anhydride 4026 ppm) was removed from the bottom of the descending stripping section of the rectification column 4. The part-stream b was fed to the common evaporator 7 of the descending stripping section and of the ascending stripping section of the rectification column 4, and a residual stream c (480 g/h, main components, in % by weight: solvent 87, acrylic acid 10, maleic anhydride 7000 ppm) was taken off from the evaporator 7 and fed to the Venturi quench 1. The vapor stream containing the crude acrylic acid and originating from the evaporator 7 was fed to the ascending stripping section of the rectification column 4 in order to recover the acrylic acid (process stage VI-II) and was purified by removal of medium boilers and high boilers by means of the acrylic acid reflux. A stream of 420 g/h of the main product acrylic acid, which still contained 1500 ppm of acetic acid and 50 ppm of maleic anhydride, was taken off at the top of the ascending stripping section of the rectification column 4. The vapor from the descending stripping section of the rectification column 4, the apparatus in which the process stage VI-I took place, was condensed as residual stream a comprising 95% by weight of acrylic acid, 0.8% by weight of solvent and 3.6% by weight of acetic acid and was likewise fed to the Venturi quench 1.

In the comparative process according to the prior art (FIG. 1), the on-stream time of the entire plant was 14 days. In contrast, virtually no soiling was found after an operating time of 24 days in the plant operated according to the novel process (FIG. 2), in particular none in the absorption column 2 and in the stripping column 3.

We claim:

1. A process for the continuous recovery of (meth)acrylic acid from the reaction gas containing (meth)acrylic acid, low boilers, medium boilers and high boilers and originating from a catalytic gas-phase oxidation by
(I) quenching the reaction gas by evaporative cooling with a high-boiling organic solvent,
(II) isolating the (meth)acrylic acid from the quenched reaction gas by absorption into the high-boiling organic solvent and
(III) separating the organic solvent laden with (meth)acrylic acid into a first part-stream (IIIA), which predominantly contains (meth)acrylic acid, and into a second part-stream (IIIB), which predominantly contains the solvent,
wherein
(IV) part-stream IIIB is stripped free of (meth)acrylic acid with inert gas,
(V) the purified solvent from part-stream IIIB is recycled to the absorption stage II and
(VI) (meth)acrylic acid from part-stream IIIA is recovered by distillation, all liquid residual streams obtained in stage VI being recycled to the quench stage I.

2. A process as claimed in claim 1, wherein the temperature in each process stage does not exceed 155°C., in particular 140°C., particularly preferably 120°C.

3. A process as claimed in claim 1, wherein the separation of the solvent laden with (meth)acrylic acid in process stage III is effected by partial evaporation.

4. A process as claimed in claim 1, wherein the recovery of the (meth)acrylic acid from part-stream IIIA by distillation is effected in the following process steps:
VI-I Separation of a residual stream (a), which, in addition to (meth)acrylic acid, contains all or virtually all of the low boilers, a part of the medium boilers and a part of the high boilers, and a part-stream (b), which contains predominantly (meth)acrylic acid and is completely or virtually completely free of low boilers, and
VI-II recovery of the (meth)acrylic acid from the part-stream (b).

5. A process as claimed in claim 4, wherein process stage VI-I is effected in a descending stripping column and process stage VI-II in an ascending stripping column.

6. A process as claimed in claim 4, wherein the part-stream (b) is separated into crude (meth)acrylic acid and a residual stream (c) in process stage VI-II.

7. A process as claimed in claim 1, wherein the stripping gas from process stage IV is recycled into the reaction gas for process stage I or II.

8. A process as claimed in claims 1, wherein the vapor from distillation stage VI-I is condensed and cooled, and the cold condensate is then recycled to process stage I or II.

9. A process as claimed in claim 1, wherein the energy for evaporating the part-stream IIIA is obtained by cooling the reaction gas.

10. A process as claimed in claim 1, wherein the stripping gas is a part-stream of the recycled gas.
